(19) United States
(12) Patent Application Publication
(10) Pub. No.: US 2015/0203063 A1
(43) Pub. Date: Jul. 23, 2015

(54) Method for avoiding an accident or for mitigating the consequences of an accident

(71) Applicant: Robert Bosch GmbH, Stuttgart (DE)

(72) Inventors: Armin KOEHLER, Sachsenheim (DE); Mariusz KOC, Stuttgart (DE)

(21) Appl. No.: 14/604,311

(22) Filed: Jan. 23, 2015

(30) Foreign Application Priority Data
Jan. 23, 2014 (DE) 10 2014 201 172.8

(51) Int. Cl. B60R 21/00; A61B 5/0402; A61B 5/18; A61B 5/0205

(52) U.S. Cl. CPC B60R 21/00; A61B 5/0205; A61B 5/0402; A61B 5/18; A61B 5/0245

(57) ABSTRACT

A method for avoiding accidents a biometric sensor system is used to ascertain vital signs of a vehicle occupant, and in the event that the vital signs lie outside a normal range, preparatory emergency measures are carried out in a safety system in the vehicle.

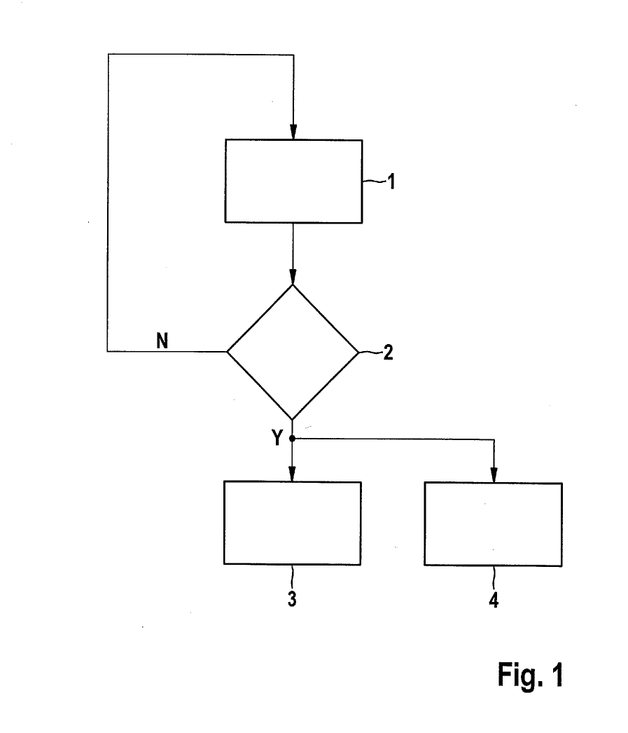

### RELATED APPLICATION INFORMATION

The present application claims priority to and the benefit of German patent application no. 10 2014 201 172.8, which was filed in Germany on Jan. 23, 2014, the disclosure of which is incorporated herein by reference.

### FIELD OF THE INVENTION

The present invention relates to a method for avoiding an accident or for mitigating the consequences of an accident, according to the definition of the species in claim 1.

### BACKGROUND INFORMATION

German patent document DE 198 01 009 C1 discusses a method in which biometric sensors in the vehicle ascertain vital signs of the driver; if an emergency or stressful situation is detected, an automatic braking action is initiated and a belt tightener is possibly actuated automatically. The biometric sensors include pressure sensors on the steering wheel, which ascertain the strength of the driver's clasp and infer the muscle contraction of the hand on this basis. Furthermore, it is also possible to use sensors for ascertaining the skin resistance, blood pressure, pulse, sweat secretion or reflexes of the pupil or an eyelid and for ascertaining the facial expression of the driver.

If the ascertained vital function exhibits a change that leads to the conclusion that an emergency or stressful situation is at hand, e.g., a strong increase in the blood pressure, then the automatic braking action and possibly the belt tightening process are initiated. In addition to the biometric sensors, the change in the position of the accelerator or brake pedal is able to be determined by sensors and utilized for assessing an emergency or stress situation.

### SUMMARY OF THE INVENTION

The present invention is based on the objective of reducing the accident or injury risk in vehicles.

According to the present invention, this objective is attained by the features described herein. The further disclosures herein indicate further refinements.

Using the method of the present invention, it is possible to avoid accidents in vehicles or at least to mitigate the accident consequences when a vehicle occupant is in a medical emergency situation. The emergency situation is ascertained with the aid of a biometric sensor system in the vehicle, which records vital signs of the vehicle occupant. The vital signs are analyzed, and in the event that the vital data lie outside a normal range, a safety-relevant intervention is carried out in the vehicle. This intervention refers to preparational emergency measures in one or in more safety system(s) in the vehicle.

The vehicle occupant in particular is the driver, but a passenger may possibly be involved as well.

This implementation offers the advantage that the preparational emergency measures are already able to be made at an early stage, even before the emergency itself has materialized. This shortens the time for implementing the preparational emergency measures if the emergency does indeed materialize, such as an accident. However, an accident could possibly also be prevented by the preparational measures, because the reaction time, e.g., for a braking intervention, is reduced. As a minimum, however, the severity of an occurring accident is less.

Conceivable as biometric sensors are, for example, electrocardiogram systems, which operate on a capacitive or inductive basis, such as sensors in the steering wheel or in the gear shift lever. Heart functions may furthermore be recorded, possibly via radar or ultrasound. In addition, facial features or expressions, the skin tone as well as certain movement patterns of the occupant can be analyzed, especially gestures and facial expressions from which the state of health of the occupant can be inferred. Also to be considered is the ascertaining of the electrical resistance of the skin, monitoring of the respiration, e.g., the respiratory rate and depth, the chemical composition of the air in the interior in order to monitor expelled respiratory air, monitoring of the brain waves or monitoring of the acceleration and pressure variables that are caused by heart activity (ballistocardiography). One or more biometric sensor(s) may be used and the data from the biometric sensors be analyzed.

These vital signs of the vehicle occupant are examined to determine whether they exceed a normal range. For instance, it can be checked whether the blood pressure of the vehicle occupant lies above a threshold value or whether the change in blood pressure exceeds a corresponding threshold value. If the vital signs lie outside the associated normal range, then a developing emergency situation of the vehicle occupant must be assumed as a minimum, whereupon the preparational emergency measures in the at least one safety system in the vehicle are taken.

According to one advantageous development, a trigger threshold of at least one safety system in the vehicle is modified. In the process, the trigger threshold of the safety system is lowered, in particular, so that an earlier or more rapid triggering of the safety system takes place as soon as a characteristic or state variable in the vehicle, possibly of the driver, drops below or exceeds an associated threshold value. The modification of the trigger threshold constitutes a preparational measure that does not yet involve the actual triggering of the safety function of the particular safety system. Nevertheless, in the event that multiple safety systems are available, it is also possible to already trigger part of the safety systems and to modify the trigger thresholds for one or more of the remaining safety system(s).

In the event of multiple safety systems in the vehicle, it is furthermore possible to mutually adapt the trigger thresholds of these safety systems. This procedure enables a stepped method with temporally sequential modifications, e.g., of trigger thresholds of different safety systems. For example, a restraining arrangement in the vehicle that represent safety systems, e.g., the belt or the vehicle seat, may be adjusted in order to stabilize the vehicle occupant, whereas a further safety system, such as an airbag, for example, is not yet triggered at this point. The triggering of the air bag, for instance, may take place only at relatively high decelerations or deformations of the vehicle body of the automobile, for which purpose the trigger thresholds may be modified, especially set to a higher value. If a looming emergency situation of the vehicle occupant is detected via the biometric sensors, the belt tightener or the driver seat is able to be activated in an effort to stabilize the vehicle occupant. The air bag, on the other hand, may still be delayed even when departing from the roadway and when encountering shocks as a result of an uneven surface, in that a higher airbag trigger threshold is set in order to trigger the airbag only at even higher forces and thus have the safety function of the airbag at one's disposal in addition.

The safety systems basically relate to all protective measures for passive safety, such as belt tighteners, airbags, an adjustable driver seat, closing of the vehicle windows and roofs, etc. Furthermore, it is also possible to influence active safety systems in the vehicle by modifying the trigger thresholds, such as brake systems, the steering system, active chassis systems or the motoric drive. To the extent that such active systems are part of a driver assistance system, the modification of the trigger thresholds makes it possible to influence the functionality of the driver assistance system, e.g., an electronic stability program (ESP).

An emergency situation is basically ascertained from vital signs that are provided by the biometric sensor system. However, in individual situations it may also be possible to take the data from a vehicle sensor system into account, via which the vehicle state is detectable. The vehicle sensor system, for example, is an inertial sensor system in the vehicle for ascertaining measured values for the linear and transverse dynamics, such as the vehicle speed, linear and transverse acceleration, as well as the rates of rotation of the wheels.

In addition, an environment sensor system may be used, via which the situation in the surroundings of the vehicle and/or the vehicle position can be registered, so that a departure from the roadway, for instance, is able to be determined. A steering-angle sensor system and a sensor system for detecting the pedal actuation in the vehicle or of other operating elements may be considered, as well. Moreover, a passenger compartment sensor system may also be taken into account, via which the weight of the driver or his/her position on the driver seat, for instance, is able to be determined.

In the event that an existing or a looming emergency situation is detected via an ascertainment of the vital signs, it is also possible to output an emergency call as an additional measure. For example, the emergency call is made to a main station in which current data from the vehicle sensor system, especially vital signs of the vehicle occupants and the position of the vehicle, are received, whereupon rescue measures are able to be initiated in the main station. Outputting the emergency call may take place prior to or following the occurrence of an accident; however, an emergency call is generated even before an accident has occurred if the biometric sensor system has detected a looming or an already existing driving incapacity of the driver.

The method runs in a closed-loop or open-loop control unit in the vehicle, which may be a component of a driver assistance system.

Further advantages and expedient embodiments may be gathered from the additional claims, the description of the drawing(s).

### BRIEF DESCRIPTION OF THE DRAWING

The FIGURE shows a flow diagram is shown for implementing the method for avoiding accidents or for mitigating the consequences of an accident.

### DETAILED DESCRIPTION

Using the method shown in the flow diagram, it is possible to avoid accidents or at least to reduce the consequences of an accident in the event that the health of the driver of a motor vehicle is suddenly compromised. To determine such a state, a biometric sensor system by which the vital signs of the driver are able to be measured is located within the vehicle. The biometric sensor system in particular involves one or more sensors for monitoring the heart activity of the driver, such as inductive or capacitive sensors for determining the electrocardiogram (EKG), the pulse, blood pressure etc. In addition or as an alternative, further biometric sensors can be provided, via which the respiratory activity or the electric skin resistance, for example, is measured.

In addition, the vehicle is advantageously equipped with a vehicle sensor system that can be used to ascertain vehicle state variables regarding the linear and transverse dynamics, such as the vehicle speed, acceleration and wheel speeds, for example. Furthermore, an environment sensor system may be provided in the vehicle, which, for instance, is able to ascertain the distance of the vehicle in relation to other vehicles on the basis of radar. The environment sensor system may also include a video-based system, for instance for ascertaining the clearance between the vehicle and the edge of the road. The vehicle is furthermore advantageously equipped with a locating system (GPS—global positioning system) in order to be able to ascertain the current vehicle location.

Method step 1 in the flow diagram symbolizes the recording of measured values in the sensor system in the vehicle which includes the biometric sensors, the vehicle sensor system for ascertaining the state variables, and the environment sensor system. The sensor system generates measured values on a continuous basis, and the vital signs supplied by the biometric sensors make it possible to infer the current state of health of the driver and possibly a passenger.

In the following method step 2, it is checked whether the vital signs lie outside a permissible normal range. Depending on the number and type of biometric sensors, different queries are able to be carried out in step 2, for instance in connection with the level of and change in the blood pressure, the heart rate and the respiratory activity. If all queried vital signs lie within the respective normal ranges, a return to the start of the method, to step 1, takes place after the no branching (“N”), and vital signs are recorded anew at regular time intervals via the sensor system.

However, if the vital signs of the driver checked in step 2 lie outside the normal range, then a current or at least developing emergency situation must be assumed, whereupon it is advanced to next method step 3 following the yes-branching (“Y”). The decision about the advancement may either be made on the basis of each polling of a vital sign value if the particular vital sign lies outside the normal range. However, it is also possible to realize a more complex query strategy and to advance to next method step 3 only if different vital signals lie outside the normal range or within defined range limits.

In the following method step 3, trigger thresholds are modified in one or multiple safety systems in the vehicle. In particular, these are passive safety systems, such as a belt tightener or an airbag system, and the threshold values may be lowered because of the worsened status of the driver's health. This has the result that the triggering of the safety system is not yet implemented; instead, only the trigger threshold is modified, so that a less severe triggering event will suffice to cause triggering of the particular safety system. Thus, it involves a measure in preparation of an emergency.

The modification of trigger thresholds in method step 3 may also mean that they are set to a higher level. For example, it may be useful to modify the trigger thresholds of different safety systems in opposite directions, such as lower the trigger threshold of the belt tightener and simultaneously increase the trigger threshold of an airbag system. This makes it possible to take driving situations into account in which the probability of an unintentional departure from the traffic lane is greater due to the worsening state of health of the driver. The higher forces that are to be expected to act on the vehicle in such a case as a result of the uneven surface can be introduced into the vehicle seat or the body structure via the activated belt tightener system. At the same time, the airbag is not yet triggered in this phase because of the increased trigger threshold; it takes place only at significantly higher forces, such as an impact against an obstacle. This procedure ensures that the airbag system as the final rescue measure is not already depleted at a point in time at which the airbag is not yet absolutely required.

In addition, it is also possible to modify trigger thresholds of active safety systems in step 3, such as brake systems, steering systems, active chassis systems or drive systems, for instance. Also conceivable are higher-level driver assistance systems such as an electronic stability program (ESP).

Parallel with the modification of trigger thresholds according to step 3, an emergency call may be transmitted according to step 4. The decision about the transmission of an emergency call may be made dependent upon the severity of the health impairment in that the emergency call is sent to a main station, where emergency measures can be initiated only in case of a significant deviation of one or more vital sign(s) from the associated normal range. Additional data may be transmitted together with the emergency call, especially in connection with the current location of the vehicle.

What is claimed is:

1. A method for avoiding an accident or for mitigating the effects of an accident, the method comprising:
ascertaining vital signs of a vehicle occupant with the aid of a biometric sensor system; and
performing an intervention in the vehicle as a function of the vital signs;
wherein in the event that the vital signs lie outside a normal range, preparatory emergency measures are carried out in a safety system in the vehicle as the intervention.

2. The method of claim 1, wherein a trigger threshold of a safety system in the vehicle is modified.

3. The method of claim 1, wherein trigger thresholds of at least two safety systems in the vehicle are adapted to each other.

4. The method of claim 2, wherein the trigger thresholds of an airbag or a belt tightener are modified.

5. The method of claim 1, wherein the safety system is an adjustable assembly of the body shell.

6. The method of claim 5, wherein the safety system is an adjustable vehicle seat.

7. The method of claim 1, wherein heart functions are measured via the biometric sensor system.

8. The method of claim 1, wherein an emergency situation is ascertained on the basis of the vital signs and additionally, on the basis of the data from a vehicle sensor system for detecting measured values that characterize the vehicle state.

9. The method of claim 1, wherein an emergency call is output if the vital signs lie outside the normal range.

10. A closed-loop or open-loop control device for avoiding an accident or for mitigating the effects of an accident, comprising:
an ascertaining arrangement to ascertain vital signs of a vehicle occupant with the aid of a biometric sensor system; and
an intervening arrangement to intervene in the vehicle as a function of the vital signs;
wherein in the event that the vital signs lie outside a normal range, preparatory emergency measures are carried out in a safety system in the vehicle as the intervention.

11. A safety system in a vehicle, comprising:
a closed-loop or open-loop control device for avoiding an accident or for mitigating the effects of an accident, including:
an ascertaining arrangement to ascertain vital signs of a vehicle occupant with the aid of a biometric sensor system; and
an intervening arrangement to intervene in the vehicle as a function of the vital signs;
wherein in the event that the vital signs lie outside a normal range, preparatory emergency measures are carried out in a safety system in the vehicle as the intervention.

12. A vehicle, comprising:
a safety system having a closed-loop or open-loop control device for avoiding an accident or for mitigating the effects of an accident, including:
an ascertaining arrangement to ascertain vital signs of a vehicle occupant with the aid of a biometric sensor system; and
an intervening arrangement to intervene in the vehicle as a function of the vital signs;
wherein in the event that the vital signs lie outside a normal range, preparatory emergency measures are carried out in a safety system in the vehicle as the intervention.
